(19) United States
(12) Patent Application Publication
(10) Pub. No.: US 2009/0305977 A1
(43) Pub. Date: Dec. 10, 2009

(54) Method for treating peripheral arterial disease with zinc finger proteins

(75) Inventors: Patrice Tremble, Tremble, CA (US); Joe Rokovich, Richmond, CA (US); Brian Annex, Richmond, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sangamo BioScience, Inc., Richmond, CA (US)

(21) Appl. No.: 12/278,967

(22) PCT Filed: Feb. 9, 2007
(86) PCT No.: PCT/US07/03538

§ 371 Date: Jul. 13, 2009

Related U.S. Application Data
(60) Provisional application No. 60/772,417, filed on Feb. 9, 2006.
(60) Provisional application No. 60/803,234, filed on May 25, 2006.

(51) Int. Cl. A61K 38/16; A61K 31/7088

(52) U.S. Cl. 514/12; 514/44 R

(57) ABSTRACT

Disclosed is a method for the administration of zinc finger proteins (ZFPs) or nucleic acids that encode such ZFPs for treating peripheral arterial disease, particularly by the repeated administration at regular intervals if such ZFPs or nucleic acids that encode such ZFPs.

### CROSS-REFERENCE TO RELATED APPLICATIONS

The present application is a nonprovisional and claims the benefit of U.S. Ser. No. 60/772,417 filed Feb. 9, 2006 and 60/803,234 filed May 25, 2006, both incorporated by reference in their entirety for all purposes.

### BACKGROUND OF THE INVENTION

It has been suggested that the development of the vascular system (sometimes referred to as the vascular tree) involves two major processes: vasculogenesis and angiogenesis. (See for example U.S. Patent Application Publication U.S. 2003/0021776 A1 to Rebar et. al which is hereby incorporated by reference in its entirety.) Vasculogenesis is the process by which the major embryonic blood vessels originally develop from early differentiating endothelial cells such as angioblasts and hematopoietic precursor cells that in turn arise from the mesoderm. Angiogenesis is the term used to refer to the formation of the rest of the vascular system that results from vascular sprouting from the pre-existing vessels formed during vasculogenesis (see, e.g., Risau et al. (1988) Devel. Biol., 125:441-450). Both processes are important in a variety of cellular growth processes including developmental growth, tissue regeneration and tumor growth, as all these processes require blood flow. Given its key role in both normal physiological and pathological processes, not surprisingly considerable research effort has been directed towards identifying. factors involved in the stimulation and regulation of angiogenesis. A number of growth factors have been purified and characterized. Such factors include fibroblast growth factors (FGFs), platelet-derived growth factor (PDGF), transforming growth factor alpha (TGF.alpha.), and hepatocyte growth factor (HGF) (for reviews of angiogenesis regulators, see, e.g., Klagsbrun et al. (1991) Ann. Rev. Physiol., 53:217-39; and Folkman et al. (1992) J. Biol. Chem., 267:10931-934). The delivery of necessary nutrients.

Thus, angiogenesis plays a critical role in a wide variety of fundamental physiological processes in the normal individual including embryogenesis, somatic growth, and differentiation of the nervous system. In the female reproductive system, angiogenesis occurs in the follicle during its development, in the corpus luteum following ovulation and in the placenta to establish and maintain pregnancy. Angiogenesis additionally occurs as part of the body's repair processes, such as in the healing of wounds and fractures. Thus, promotion of angiogenesis can be useful in situations in which establishment or extension of vascularization is desirable. Angiogenesis, however, is also a critical factor in a number of pathological processes, perhaps must notably tumor growth and metastasis, as tumors require continuous stimulation of new capillary blood vessels in order to grow. Other pathological processes affected by angiogenesis include conditions associated with blood vessel proliferation, especially in the capillaries, such as diabetic retinopathy, arthropathies, psoriasis and rheumatoid arthritis.

Rebar et. al (cited above) also disclose a variety of zinc finger proteins (ZFPs) for use in regulating gene expression. Certain of the ZFPs are designed to bind to specific target sequences within genes and thereby modulate the expression of these genes. The ZFPs can be fused to a regulatory domain as part of a fusion protein. By selecting either an activation domain or repressor domain for fusion with the ZFP, one can either activate or repress gene expression. Thus, by appropriate choice of the regulatory domain fused to the ZFP, one can selectively modulate the expression of a gene and hence various physiological processes correlated with such genes. Thus, with angiogenesis, for example, by attaching an activation domain to a ZFP that binds to a target sequence within a gene that affects angiogenesis, one can enhance certain beneficial aspects associated with angiogenesis (e.g., alleviation of ischemia). In contrast, if angiogenesis is associated with harmful processes (e.g., delivery of blood supply to tumors) one can reduce angiogenesis by using ZFPs that are fused to a repressor. Hence, binding of this type of ZFP to a gene involved in angiogenesis can significantly reduce angiogenesis.

Rebar et. al further describe a family of endothelial cell-specific growth factors, the vascular endothelial growth factors (VEGFs), together with their cognate receptors, are primarily responsible for stimulation of endothelial cell growth and differentiation. These factors are members of the PDGF family and appear to act primarily via receptor tyrosine kinases (RTKs).

The first identified and most well studied member of this particular family is the vascular endothelial growth factor (VEGF), also referred to as VEGF-A. This particular growth factor is a dimeric glycoprotein in which the two 23 kD subunits are joined via a disulfide bond. Five VEGF-A isoforms encoded by distinct mRNA splice variants appear to be equally effective in stimulating mitogenesis in endothelial cells, but tend to have differing affinities for cell surface proteoglycans.

VEGF-A acts to regulate the generation of new blood vessels during embryonic vasculogenesis and then subsequently plays an important role in regulating angiogenesis later in life. Studies showing that inactivation of a single VEGF-A allele results in embryonic lethality provide evidence as to the significant role this protein has in vascular development and angiogenesis (see, e.g., Carmeliet et al. (1996) Nature 380: 435-439; and Ferrara et al. (1996) Nature, 380: 439-442). VEGF-A has also been shown to have other activities including a strong chemoattractant activity towards monocytes, the ability to induce the plasminogen activator and the plasminogen activator inhibitor in endothelial cells, and to induce microvascular permeability. VEGF-A is sometimes also referred to as vascular permeability factor (VPF) in view of this latter activity. The isolation and properties of VEGF-A have been reviewed (see, e.g., Ferrara et at. (1991) J. Cellular Biochem. 47: 211-218; and Connolly, J. (1991) J. Cellular Biochem. 47:219-223).

### SUMMARY OF THE INVENTION

The present inventors have found that administration of ZFPs, or nucleic acids that encode such ZFPs, are useful for treating diseases in which an increase in perfusion or capillary density are beneficial, for example, peripheral arterial disease, by means of a multiple dosing regimen. It has, moreover, been found that administration of an effective amount of a zinc finger protein or a nucleic acid that encodes a zinc finger protein in a multiple dosing regimen induces a more stable angiogenic response, measured as an increase in capillary density, increase in perfusion, or an increase in oxidative fibers in injected muscle. It has, moreover, been found that a more stable angiogenic response and increased capillary density in a patient can be enhanced by administering to the patient in a multiple dosing regimen, an effective amount of a zinc finger protein or a nucleic acid that encodes a zinc finger protein. It has additionally been found that an increase in stem cells, such as bone marrow vascular progenitor cells and dendritic or monocytic precursor cells, which are mobilized in the peripheral blood circulation, occurs after administration of an effective amount of a zinc finger protein or a nucleic acid that encodes a zinc finger protein in a multiple dosing regimen. These discoveries provide the basis for a new treatment of diseases, including cardiovascular diseases, characterized by an impaired oxidative capacity of skeletal muscle, by administering an effective amount of a zinc finger protein or a nucleic acid that encodes a zinc finger protein.

The invention further provides a method of stimulating angiogenesis in a patient. The method comprises repeatedly administering to a patient in need of stimulation of angiogenesis a zinc finger protein that induces expression of VEGF-A, or a nucleic acid encoding the zinc finger protein, whereby expression of VEGF-A mobilizes bone marrow vascular progenitor cells and/or dendritic or monocytic precursor cells from the bone marrow to the peripheral circulation, whereby the cells are distributed to stimulate angiogenesis at sites in the patient disseminated from the site or sites of administration. Optionally, the zinc finger protein or nucleic acid encoding the same is repeatedly administered by localized administration. Optionally, the patient is suffering from peripheral arterial disease, and the cells are distributed to treat the disease at disseminated sites in the patient. Optionally, the repeatedly administering step comprises repeatedly administering the zinc finger protein or nucleic acid encoding the same by injection into a muscle of the patient. Optionally, the zinc finger protein or nucleic acid encoding the same is repeatedly injected into the same muscle of the patient. Optionally, the zinc finger protein or nucleic acid encoding the zinc finger protein is repeatedly injected at the same site or sufficiently proximal sites so that some cells receive repeated administrations of the nucleic acid. Optionally, the muscle is non-ischemic.

The invention further provides for use of a zinc finger protein that induces expression of VEGF-A or a nucleic acid encoding the same in the manufacture of a medicament to stimulate angiogenesis at sites in the patient disseminated from a localized site of repeated administration. Optionally, the zinc finger protein or nucleic acid encoding the same is formulated for intramuscular administration. Optionally, the use is to treat peripheral arterial disease.

### BRIEF DESCRIPTION OF THE DRAWINGS

FIGS. 1A-D show H&E stained sections of rat skeletal muscle surrounding the injection site retrieved 14 days after the first injection with vehicle or EW-A-401. Tissue from animals that were injected with vehicle (A, C) or EW-A-401 (B, D) once (A, B) or twice (C, D). The bar represents 100 um. The intracellular white splotches represent artifacts of tissue freezing.

FIGS. 2A-F show H&E stained sections of rat skeletal muscle surrounding the injection site retrieved 28 days after the first injection with vehicle or EW-A-401. Tissue from animals that were injected with vehicle (A, C, E) or EW-A-401 (B, D, F) once (A, B), twice (C, D), or four times (E, F). The bar represents 100 μm. The intracellular white splotches represent artifacts of issue freezing.

FIGS. 3A-D show increased capillary density in animals treated with EW-A-401 is suggested by alkaline phosphatase-positive cells. Skeletal muscle surrounding the injection site in rats treated with vehicle (A, C) or EW-A-401 (B, D) receiving one (A, B) or two (C, D) doses was harvested 14 days following the initiation of treatment. The dark spots at the myofibers' edge indicate cells positive for alkaline phosphatase activity, a marker for endothelial cells. The bar represents 50 um.

FIGS. 4A-F show increased capillary density in animals treated with multiple doses of EW-A-401 as indicated by alkaline phosphatase—positive cells. Tissue from animals that were injected with vehicle (A, C, E) or EW-A-401 (B, D, F) once (A, B), twice (C, D), or four times (E, F). The bar represents 50 um.

FIG. 5A-D show serial sections of muscle harvested at 14 D from animals receiving two doses of EW-A-401 were stained with anti-CD31 monoclonal antibody (A, B) or with an alkaline phosphatase stain (C, D). CD31 and alkaline phosphatase staining is indicated by dark spots. Panels A and C present a lower magnification view of staining in aligned sections; a view at higher magnification of the boxed area is provided in panels B and D. Note the similarity in staining pattern. The bar in panels A and C represents 100 um.

FIGS. 6A and B show the ratio of cells positive for alkaline phosphatase (AP) to muscle fibers in Rat tissue at 14 or 28 D. Panel A plots the ratio of cells staining positive for alkaline phosphatase divided by the number of myofibers in examined tissue sections from animals sacrificed 14 D following the initial injection. Panel B plots the same ratio from animals receiving one, two or four doses of EW-A-401, harvested 28 D after the initial dose.

### DETAILED DESCRIPTION OF THE INVENTION

### 1. Definitions

Various terms and techniques utilized in molecular biology, recombinant DNA, and related fields are now well-known to those of skill in the art. Useful techniques, some of which are useful for the present invention, are discussed, for example, in Sambrook et al., Molecular Cloning: A Laboratory Manual, (2^{nd} ed. 1999), Ausubel et al., Current Protocols In Molecular Biology (1994). The following terms are particularly relevant to the present invention.

Chimeric: The term “chimeric” is used to describe genes, as defined supra, or constructs wherein at least two of the elements of the gene or construct, such as the promoter and the coding sequence and/or other regulatory sequences and/or filler sequences and/or complements thereof, are heterologous to each other.

Domain: Domains are fingerprints or signatures that can be used to characterize protein families and/or parts of proteins. Such fingerprints or signatures can comprise conserved (1) primary sequence, (2) secondary structure, and/or (3) three-dimensional conformation. Generally, each domain has been associated with either a family of proteins or motifs. Typically, these families and/or motifs have been correlated with specific in-vitro and/or in-vivo activities. A domain can be any length, including the entirety of the sequence of a protein. Detailed descriptions of the domains, associated families and motifs, and correlated activities of the polypeptides of the instant invention are described below. Usually, the polypeptides with designated domain(s) can exhibit at least one activity that is exhibited by any polypeptide that comprises the same domain(s).

Effective amount (or “therapeutically effective” amount): These terms refer to the amount of a compound, agent or pharmaceutical composition that is sufficient, but nontoxic, to provide the desired effect. The term refers to an amount sufficient to treat a subject, typically a human subject but also any mammal or animal. Thus, the term therapeutic amount refers to an amount sufficient to remedy or otherwise treat a particular disease state or symptoms, by preventing, hindering, retarding, reducing, ameliorating or reversing the progression of the disease.

Nucleic acid capable of expressing a ZFP: This term, as used herein, designates a nucleic acid which comprises a polynucleotide sequence encoding a ZFP which is operably linked to a transcriptional control sequence so as to ensure transcription in the target cells. According to the present invention, said “nucleic acid” can be a fragment or a portion of a polynucleotide sequence, without size limitation, which may be either linear or circular, natural or synthetic, modified or not (see U.S. Pat No. 5,525,711, U.S. Pat. No. 4,711,955, U.S. Pat. No. 5,792,608 or EP 302175 for modification examples). Depending on the considered sequence, it may be, inter alia, a genomic DNA, a cDNA, a mRNA or a synthetic DNA. The nucleic acid sequence can be homologous or heterologous to the host cells. The nucleic acid can be in the form of plasmid DNA and the polynucleotide can be a naked plasmid DNA. A wide range of plasmids is commercially available and well known by one skilled in the art. These available plasmids are easily modified by standard molecular biology techniques (e.g., Sambrook et al, 1989, Molecular cloning, A Laboratory Manual, Cold Spring Harbor Laboratory Press, Cold Spring Harbor, N.Y.). Plasmids derived from pBR322 (Gibco BRL), pUC (Gibco BRL), pBluescript (Stratagene), pREP4, pCEP4 (Invitrogen) and also pPoly (Lathe et al., 1987, Gene 57, 193-201) are illustrative of these modifications.

The nucleic acid may be encoded by virus

Percentage of sequence identity: “Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity compared to a reference sequence using the programs.

Plasmid: The term plasmid refers to a circular, double-stranded unit of DNA that replicates within a cell independently of the chromosomal DNA. Plasmids are most often found in bacteria and are used in recombinant DNA research to transfer genes between cells.

Promoter: The term “promoter,” as used herein, refers to a region of sequence determinants located upstream from the start of transcription of a gene and which are involved in recognition and binding of RNA polymerase and other proteins to initiate and modulate transcription. A basal promoter is the minimal sequence necessary for assembly of a transcription complex required for transcription initiation. Basal promoters frequently include a “TATA box” element usually located between 15 and 35 nucleotides upstream from the site of initiation of transcription. Basal promoters also sometimes include a “C CAAT box” element (typically a sequence CCAAT) and/or a GGGCG sequence, usually located between 40 and 200 nucleotides, preferably 60 to 120 nucleotides, upstream from the start site of transcription. “Constitutive” promoters actively promote transcription under most, but not necessarily all, environmental conditions and states of development or cell differentiation.

Regulatory Sequence: The term “regulatory sequence,” as used in the current invention, refers to any nucleotide sequence that influences transcription or translation initiation and rate, and stability and/or mobility of the transcript or polypeptide product. Regulatory sequences include, but are not limited to, promoters, promoter control elements, protein binding sequences, 5′ and 3′ UTRs, transcriptional start site, termination sequence, polyadenylation sequence, introns, certain sequences within a coding sequence, etc.

Signal Peptide: A “signal peptide” as used in the current invention is an amino acid sequence that targets the protein for secretion, for transport to an intracellular compartment or organelle or for incorporation into a membrane. Signal peptides are indicated in the tables and a more detailed description located below.

Stringency: “Stringency” as used herein is a function of probe length, probe composition (G+C content), and salt concentration, organic solvent concentration, and temperature of hybridization or wash conditions. Stringency is typically compared by the parameter Tₘ, which is the temperature at which 50% of the complementary molecules in the hybridization are hybridized, in terms of a temperature differential from Tₘ. High stringency conditions are those providing a condition of Tₘ-5° C. to Tₘ-10° C. Medium or moderate stringency conditions are those providing Tₘ-20° C. to Tₘ-29° C. Low stringency conditions are those providing a condition of Tₘ-40° C. to Tₘ-48° C. The relationship of hybridization conditions to Tₘ (in ° C.) is expressed in the mathematical equation

Tₘ=81.5−16.6(log₁₀[Na⁺])+0.41 (%G+C)−(600/N) (1)

where N is the length of the probe. This equation works well for probes 14 to 70 nucleotides in length that are identical to the target sequence. The equation below for Tₘ of DNA-DNA hybrids is useful for probes in the range of 50 to greater than 500 nucleotides, and for conditions that include an organic solvent (formamide).

Tₘ=81.5+16.6 log {[Na⁺]/(1+0.7[Na⁺])}+0.41 (%G+C)−500/L 0.63(%formamide) (2)

where L is the length of the probe in the hybrid. (P. Tijessen, “Hybridization with Nucleic Acid Probes” in Laboratory Techniques in Biochemistry and Molecular Biology, P. C. vand der Vliet, ed., c. 1993 by Elsevier, Amsterdam.) The Tₘ of equation (2) is affected by the nature of the hybrid; for DNA-RNA hybrids Tₘ is 10-15° C. higher than calculated, for RNA-RNA hybrids Tₘ is 20-25° C. higher. Because the Tₘ decreases about 1° C. for each 1% decrease in homology when a long probe is used (Bonner et al., J. Mol. Biol. 81:123 (1973)), stringency conditions can be adjusted to favor detection of identical genes or related family members.

Equation (2) is derived assuming equilibrium and therefore, hybridizations according to the present invention are most preferably performed under conditions of probe excess and for sufficient time to achieve equilibrium. The time required to reach equilibrium can be shortened by inclusion of a hybridization accelerator such as dextran sulfate or another high volume polymer in the hybridization buffer.

Stringency can be controlled during the hybridization reaction or after hybridization has occurred by altering the salt and temperature conditions of the wash solutions used. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

Target site: In the context of the present invention, “target site” is the nucleic acid sequence recognized by a ZFP. A single target site typically has about four to about ten base pairs. Typically, a two-fingered ZFP recognizes a four to seven base pair target site, a three-fingered ZFP recognizes a six to ten base pair target site, and a six fingered ZFP recognizes two adjacent nine to ten base pair target sites

Translational start site: In the context of the current invention, a “translational start site” is usually an ATG in the cDNA transcript, more usually the first ATG. A single cDNA, however, may have multiple translational start sites.

Transcription start site: “Transcription start site” is used in the current invention to describe the point at which transcription is initiated. This point is typically located about 25 nucleotides downstream from a TFIID binding site, such as a TATA box. Transcription can initiate at one or more sites within the gene, and a single gene may have multiple transcriptional start sites, some of which may be specific for transcription in a particular cell-type or tissue.

Untranslated region (UTR): A “UTR” is any contiguous series of nucleotide bases that is transcribed, but is not translated. These untranslated regions may be associated with particular functions such as increasing mRNA message stability. Examples of UTRs include, but are not limited to polyadenylation signals, terminations sequences, sequences located between the transcriptional start site and the first exon (5′ UTR) and sequences located between the last exon and the end of the mRNA (3′ UTR).

Variant: The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc).

VEGF: The term “VEGF gene” refers generally to any member of the VEGF family of genes as described supra or collection of genes from the VEGF family having a native VEGF nucleotide sequence, as well as variants and modified forms regardless of origin or mode of preparation. The VEGF genes can be from any source. Typically, the VEGF genes refer to VEGF genes in mammals, particularly humans. A VEGF gene having a native nucleotide sequence is a gene having the same nucleotide sequence as a VEGF gene as obtained from nature (i.e., a naturally occurring VEGF gene). More specifically, the term includes VEGF-A (including the isoforms VEGF-A121, VEGF-A145, VEGF-A165, VEGF-A189, and VEGF-A206); VEGF-B (including the isoforms VEGF-B167, and VEGF-B186); VEGF-C; VEGF-D; VEGF-E (various VEGF-like proteins from virus strains as described in the Background section); VEGF-H; VEGF-R; VEGF-X; VEGF-138; and P1GF (both P1GF-1 and P1GF-2). The term also includes variants of specific isoforms. For example, the term includes not only the isoform VEGF-145, but also VEGF-145-I, VEGF-145-II, and VEGF-145-III. The term also encompasses allelic variants, other isoforms resulting from alternative exon splicing, forms that are functionally equivalent to native sequences, and nucleic acids that are substantially identical to a native VEGF gene.

Zinc finger protein or “ZFP”: These terms refer to a protein having DNA binding domains that are stabilized by zinc. Such proteins have areas with regularly spaced cysteine amino acids that appear to be involved in binding zinc atoms. The individual DNA binding domains are typically referred to as “fingers”. A ZFP has least one finger, typically two, three, four, five, six or more fingers. Each finger binds from two to four base pairs of DNA, typically three or four base pairs of DNA (often referred to as a “subsite”). A ZFP binds to a nucleic acid sequence called a target site or target segment. Each finger typically comprises an approximately 30 amino acid, zinc-chelating, DNA-binding subdomain. An exemplary motif characterizing one class of these proteins (C2H2 class) is -Cys-(X)2-4-Cys-(X)12-His-(X)3-5-His (where X is any amino acid) (SEQ ID NO:208). Additional classes of zinc finger proteins are known and are useful in the practice of the methods, and in the manufacture and use of the compositions disclosed herein (see, e.g., Rhodes et al. (1993) Scientific American 268:56-65). Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)). As utilized herein, the term ZFPs is sometimes utilized to indicate either/or the zinc finger protein or a nucleic acid encoding the same.

### 2. Zinc Finger Proteins Useful in the Invention

Zinc finger proteins are comprised of domains including, but not limited to, at least one zinc finger DNA binding domain, and, frequently, one or more transcriptional activation domains. Various zinc finger proteins, and the nucleic acids which encode the same, are useful for the present invention to stimulate the pharmacologically important results described herein. One such family of ZFPs has zinc finger DNA binding domains of the amino acid sequences DRSNLTR, TSGHLTR, and/or RSDHLSR. This family of ZFPs specifically binds to the DNA sequence, GGGGGTGAC, and said DNA sequence is present in the regulatory sequences of mammalian VEGF.

As described in the examples and incorporated references below, once the ZFPs of the present invention are bound to VEGF regulatory sequences, their transcriptional activation domains upregulate the expression of the VEGF gene, thereby stimulating angiogenesis. In other embodiments of the present invention, other ZFPs may be utilized to stimulate angiogenesis, such as those described in U.S. Patent Publication 2003/0021776A1, which is hereby incorporated by reference in its entirety. Yet other embodiments of the present invention involve the administration of nucleic acids that code for fusion proteins comprising the zinc finger DNA binding domains described above filsed to transcriptional repression domains (for example, the engrailed repressor). Once expressed in vivo, said fusion proteins will bind VEGF regulatory sequence and repress VEGF expression, thereby inhibiting angiogenesis.

### 3. Preparation of Zinc Finger Proteins

ZFPs both polypeptides and nucleotides, can be prepared by synthetic methods well known to those skilled in the art, and described for example, in Sambrooks et al (above).

### 4. Compositions Containing and Administration of ZFPs

Administration according to the invention is accomplished by administering to the patient either the zinc finger protein or a vector encoding the nucleic acid molecule that encodes the same or the nucleic acid molecule that encodes the same . For administration of the protein, the protein is typically administered as a therapeutically effective amount in a pharmaceutical composition in combination with a pharmaceutically acceptable carrier or diluent or excipient. The present invention also provides pharmaceutical compositions containing a pharmaceutically effective amount of a ZFP in combination with one or more pharmaceutically acceptable carriers, excipients, diluents or adjuvants. For example, the ZFP may be formulated in the form of tablets, pills, powder mixtures, capsules, injectables, solutions, suppositories, emulsions, dispersions, food premix, and in other suitable forms. They may also be manufactured in the form of sterile solid compositions, for example, freeze-dried and, if desired, combined with other pharmaceutically acceptable excipients. Such solid compositions can be reconstituted with sterile water, physiological saline, or a mixture of water and an organic solvent, such as propylene glycol, ethanol, and the like, or some other sterile injectable medium immediately before use of parenteral administration.

Typical pharmaceutically acceptable carriers are, for example, manitol, urea, dextrans, lactose, non-reducing sugars, potato and maize starches, magnesium stearate, talc, vegetable oils, polyalkylene glycols, ethyl cellulose, poly(vinyl-pyrrolidone), calcium carbonate, ethyloleate, isopropyl myristate, benzyl benzoate, sodium carbonate, gelatin, potassium carbonate, silicic acid. The pharmaceutical preparation may also contain non toxic auxiliary substances such as peptides, emulsifying, preserving, wetting agents, and the like as for example, sorbitan monolaurate, triethanolamine oleate, polyoxyethylene monostearate, glyceryl tripalmitate, dioctyl sodium sulfosuccinate, and the like.

To obtain expression of a cloned nucleic acid encoding a ZFP, a chimeric zinc finger protein is typically subcloned into a virus or expression vector that contains a promoter to direct transcription. Suitable bacterial and eukaryotic promoters are well known in the art and described, e.g., in Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). Bacterial expression systems for expressing the zinc finger protein are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known in the art and are also commercially available.

The promoter used to direct expression of a chimeric zinc finger protein nucleic acid depends on the particular application. For example, a strong constitutive promoter is typically used for expression and purification of zinc finger protein. In contrast, when a zinc finger protein is administered in vivo for gene regulation, either a constitutive or an inducible promoter is used, depending on the particular use of the zinc finger protein. The promoter typically can also include elements that are responsive to transactivation, e.g., hypoxia response elements, Gal4 response elements, lac repressor response element, and small molecule control systems such as tet-regulated systems and the RU-486 system (see, e.g., Gossen & Bujard, Proc. Natl. Acad. Sci. U.S.A. 89:5547 (1992); Oligino et al., Gene Ther. 5:491-496 (1998); Wang et al., Gene Ther. 4:432-441 (1997); Neering et al., Blood 88:1147-1155 (1996); and Rendahl et al, Nat. Biotechnol. 16:757-761 (1998)).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the zinc finger protein, and signals required, e.g., for efficient polyadenylation of the transcript, transcriptional termination, ribosome binding sites, or translation termination. Additional elements of the cassette may include, e.g., enhancers, and heterologous spliced intronic signals.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the ZFP protein of choice.

In one embodiment, the transcriptional control sequence comprises a promoter element. Preferably, one would use a high expression promoter. Such a promoter may be for example selected from the group consisting of viral promoters and muscle specific promoters, or a combination thereof. Examples of such viral promoters are the SV40 early and late promoters, the adenovirus major late promoter, the Rous Sarcoma Virus (RSV) promoter, the Cytomegalovirus (CMV) immediate-early promoter, the herpes simplex virus (HSV) promoter, the MPSV promoter, the 7.5 k promoter, the vaccinia promoter and the Major-intermediate-early (MIE)promoter. Examples of muscle specific promoters are the smooth muscle 22 (SM22) promoter, the myosin light chain promoter, the myosin heavy chain promoter, the skeletal alpha-actin promoter and the dystrophin promoter. The Cytomegalovirus (CMV) immediate-early promoter is used in the Example below The natural promoter of the beta-interferon encoding sequence might also be used (U.S. Pat. No. 4,738,931). The polynucleotide sequence of the promoter can be a naturally occurring promoter sequence isolated from biological nucleic acid material or chemically synthesized. The promoter sequence can also be artificially constructed by assembling elements previously screened for transcriptional activity leading to potencies which can exceed those of naturally occurring ones (Li et al., 1999, Nature Biotech., 17, 241-245).

The expression cassette (including the ZFP coding sequence and promoter) can be constructed using routine cloning techniques known to persons skilled in the art (for example, see Sambrook et al., 1989, supra).

In still another aspect of the invention, the transcriptional control sequence further comprises at least one enhancer element. The term “enhancer” refers to a regulatory element which activates transcription in a position and orientation independent way. Several enhancer elements have been identified to date in many genes. For example, the enhancer element may be a myosin light chain enhancer. More preferably, the enhancer used in the expression cassette of the present invention is of vertebrate origin, more preferably of mammalian origin. The rat myosin light chain ⅓ enhancer (Donoghue et al., 1988, Gene & Dev., 2, 1779-1790) is especially useful. The enhancer element is operably linked to the promoter, may be localized either upstream or downstream of said promoter and may be used in either orientation. According to another embodiment, the transcriptional control sequence comprises several enhancer sequences, the sequences of which are identical or selected independently of one another. The transcriptional control sequence may further comprise at least one sequence ensuring the polyadenylation of the transcribed RNA molecules. Such a sequence may be selected from the group consisting of the bGH (bovine growth hormone) polyadenylation signal (EP 173552), the SV40 polyadenylation signal and the globine polyadenylation signal, and is generally located at the 3′-end of the sequence encoding beta-interferon.

The pharmaceutical composition described above can be administered by any suitable route. Administration into vertebrate target tissues, and more specifically into the muscle, can be performed by different delivery routes (systemic delivery and targeted delivery). According to the present invention, the pharmaceutical composition is preferably administered into skeletal muscle, however administration can also occur in other tissues of the vertebrate body including those of non-skeletal muscle. Similarly, the nucleic acid can be associated with targeting molecules which are capable to direct its uptake into targeted cells. Gene therapy literature provides many mechanisms for efficient and targeted delivery and expression of genetic information within the cells of a living organism. Administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol routes. Injection, and specifically intramuscular injection, is preferred.

Preferably, the concentration of the nucleic acid in the pharmaceutical composition is from about 0.1 μg/ml to about 20 mg/ml, particularly about 2 mg/ml, to about 10 mg /ml.

The active (i.e., therapeutically effective) dose, or the amount of nucleic acid which should be injected for obtaining satisfactory amount of the ZFP, is from about 1 μg to 1 g, or from about 1 mg to 1 g, or from about 1 mg to 100 mg, generally, a maximum dose of 40 mg/70 kg person. Preferably, the maximum single dose is 80 mg of DNA administered. The separate administrations can be performed by different delivery routes (systemic delivery and targeted delivery, or targeted deliveries for example). In a preferred embodiment, each delivery should be done into the same target tissue and most preferably by injection.

The administered volume preferably varies from about 10 μl to 500 ml, most preferably from about 100 μl to 100 ml. The administered volume can be adapted depending on the administration route, the treated patient and the patient's weight.

The present invention further relates to a kit comprising a nucleic acid capable of expressing a ZFP and a delivery tool. Preferably, the nucleic acid is in solution in a pharmaceutically acceptable carrier. In a preferred embodiment, the nucleic acid is a nucleic acid as described herein above in connection with the use according to the invention. The kit is intended for gene transfer, especially for the treatment of the human or animal body, and in particular for the treatment of a disease.

The present invention also relates to a method for treating a cardiovascular disease in a mammal which comprises administering to said mammal an effective amount of a nucleic acid encoding a ZFP operably linked to a promoter to result in expression of the protein when delivered to a tissue of the mammal. The expression of the ZFP protein results in an improvement of the clinical status of the treated mammal.

In addition, the present invention also relates to a method for increasing the number of stem cells, particularly bone marrow vascular progenitor cells and/or dendritic or monocyte precursor cells, in a mammal and mobilizing them to the peripheral blood circulation which comprises administering to said mammal an effective amount of a nucleic acid encoding a ZFP operably linked to a promoter to result in expression of the protein when delivered to a tissue of the mammal.

Conventional viral and non-viral based gene transfer methods can be used to introduce nucleic acids encoding the present ZFPs in mammalian cells or target tissues. Such methods can be used to administer nucleic acids encoding ZFPs to cells in vitro. In some instances, the nucleic acids encoding ZFPs are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids encoding the ZFPs provided herein include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam.™. and Lipofectin.™.). Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration).

The nucleic acids of the invention can be administered in a variety of ways, including naked or non-naked form. “Naked” means that said nucleic acid, irrespective of its nature (DNA or RNA), its size, its form (for example single/double stranded, circular/linear), etc. is defined as being free from association with transfection-facilitating agents (e.g. viral particles, liposomal formulations, charged lipids, peptides, polymers, or precipitating agents). (Wolf et al., 1990, Science 247, 1465-1468; EP 465529). “Non-naked” means that said nucleic acid may be associated with (i) viral proteins and/or polypeptides forming what is usually called a virus (e.g. adenovirus, retrovirus, poxvirus, etc.); or (ii) forming a complex in which the nucleic acid is complexed with but not included in viral elements such as viral capsid proteins (See, e.g., U.S. Pat. No. 5,928,944 and WO 9521259); or (iii) with any agent which can participate in the transfer and/or uptake of said nucleic acid into cells.

One such transfer and/or uptake agent is poloxamer, also known as Pluronic® (available from BASF) or Synperonic. Poloxymer is a block copolymer of ethylene oxide and propylene oxide available in several types, including poloxymer 124, poloxymer 188, poloxymer 237, poloxymer 338, and poloxymer 407, and PE6400. In aqueous solutions, individual poloxymer molecules, referred to as “unimers,” form a molecular dispersion when present in concentrations below the critical micellular concentration (CMC). When present in aqueous solutions at or above their CMC, poloxymer unimers assemble into micelles having hydrophobic cores, hydrophilic shells, and a variety of structural morphologies (e.g. spheres, rods, lamella, and cylinders). (Kabanov et al., 2002, Advanced Drug Delivery Reviews 55, 223-233).

In vivo studies have shown that, unlike nucleic acids administered in naked form, nucleic acids administered in poloxymer formulations efficiently enter cells, where the nucleic acids of said formulations direct the expression of therapeutic proteins at levels useful for gene therapy (Lemieux et al., 2000, Gene Therapy 7, 986-999). Because maximal gene therapy activity is typically obtained from nucleic acid-poloxymer formulations wherein the poloxymer concentration is close to its CMC, it is has been hypothesized that both poloxymer micelles and unimers play important roles in promoting said activity (Kabanov et al., 2002, Advanced Drug Delivery Reviews 55, 223-233).

### 5. Pharmacological/Therapeutic Effects of the Invention

As noted above, present inventors have found that administration of ZFPs, or nucleic acids that encode such ZFPs, are useful for treating peripheral arterial disease by stimulating a more stable angiogenic response, an increase in capillary density, and an increase in tissue perfusion in a patient.

The present inventors have also found that administration of ZFPs, or nucleic acids that encode such ZFPs, are useful for increasing the number of bone marrow vascular progenitor cells and/or dendritic or monocyte precursor cells in a mammal and mobilizing them to the peripheral blood circulation.

It has been particularly determined according to the present invention that administration of ZFPs is effective when administered in an effective amount in a repeated dosage regimen. As noted above, it was observed in normal animals that a simple, single dose of ZFPs provided some beneficial response, but the observed beneficial effects tended to decline at later time points (e.g. 28 days). As compared to a single dosage administration, the present inventors have found that repeated administration of a suitable dosage amount of a ZFP at an interval of two or more days, such as on each of 0, 4, 7 or 10 days, provides beneficial results to a patient. Suitable dosage amounts are provided to a patient in an amount of 0.1 to 20 mg/ml of nuclei acid per dose or 1 to 100 mg of nucleic acid per 75 kg of body weight per dose. Administration of repeated (more than one) dosages at intervals of 1, 4, 7 and 10 days provides for a more stable angiogenic response. Improved response, as compared to a single dose, can be achieved by administering repeated dosages to a patient, repeated at a variety of intervals, spaced from 2 to 30, or more, days and administering to the patient a total of 2 or more doses, including up to 2-4, 2-8, 2-10, or more total doses. Improved results can be achieved by establishing a repeated dosage regimen wherein the patient receives an additional, repeated dosage on an established interval, for example, 30 days, monthly or other spaced intervals, whereby the patient receives more than one dose wherein the individual doses are repeated at intervals spaced apart by 2 or more days.

### 6. In Vivo Experiments According to the Invention—Example 1

6.1. Experimental Design and Objectives

The usefulness of the invention was shown by in vivo experiments to establish that
1. Administration of repeated doses of a vector containing a ZFP, induces more durable angiogenic response in skeletal muscle of normal animals.

A gene-transfer based therapy is designed to induce therapeutic angiogenesis for applications in peripheral cardiovascular disease, utilizing a plasmid expression vector (EW-A-401) that encodes an engineered zinc finger transcription factor (32E-ZFP) that regulates the expression of the endogenous VEGF-A gene. In vitro, EW-A-401 leads to the expression of 32E-ZFP, the active moiety in EW-A-401 which targets unique sequences in the promoter of VEGF-A leading to selective increases in expression of the VEGF-A gene. (Liu, P.Q, et al., J Biol Chem. 276:11323-34.; Rebar, E. J. et al., (2002) Nature Medicine 8: 1427-1432). This specific, targeted increase in VEGF-A gene expression includes increases in the major splice variants of VEGF-A, which contrasts with studies in which gene transfer vectors encoding cDNAs of single isoforms of VEGF-A (reviewed in Rebar et al. 2002). In vivo, the transgene of EW-A-401 induces an increase in capillary density in normal rodents and rabbits (Rebar et al., 2002) and in disease models of hind limb ischemia (Dai, Q. et al., (2004) Circulation, 110:2467-2475). Work to date has evaluated the efficacy of EW-A-401 in driving an angiogenic response in response to a single dose injected into skeletal muscle.

The following experiments were designed to evaluate repeated dosing of EW-A-401 in healthy animal models. The repeated dosing interval of four days in this study was derived from a previous study showing expression of the transgene peaking at three days and falling rapidly thereafter. Endpoints in this study include capillary density (measured after alkaline phosphatase or CD31 staining), inflammation (hematoxalin and eosin staining), and analysis of myofiber phenotype.
6.2. Materials and Methods

### Test Articles

EW-A-401: The vector comprises a plasmid encoding 32E-ZFP formulated in 1% P407, 150 mM NaCl, 2 mM Tris pH 8.0, to a final concentration of2 mg/ml plasmid. The transgene cassette is specifically comprised of a structure of CMV promoter—a nuclear localization sequence-ZFP sequence—activation domain-polyadenylation sequence.

The promoter is a cytomegalovirus (CMV) promoter and the polyA sequences are bovine growth hormone sequences.

The zinc finger protein is specifically nominated 32E-ZFP, and has a structure capable of binding to the VEGF target GGGGGTGAC, and has one or more of the zinc fingers of the sequences DRSNLTR, TSGHLTR and RSDHLSR.

Vehicle Control: A solution of 1%, p407, 150 mM NaCl, 2 mM Tris pH 8.0.

### Injection Volume

The injection volume per limb of either EW-A-401 or Vehicle Control was 0.1 mL for rats and 3.08 mL in rabbits. Rats received a single injection in the limb; while rabbits received five injections per dose in the limb (spaced evenly across the muscle).

### Animal Model

This study was performed in the normal hind limb of Sprague Dawley rats. Each animal received from one to four doses of test or control article into the rectus femoris (RF) muscle. The injection site was identified with a suture, and the same injection site was used for repeated doses Table 1 lists and describes the treatment groups.

TABLE 1
Rat Treatment Groups

| | Study Day | | | Animals per |
|---|---|---|---|---|
| Group | Dosing(s) | Harvest | Treatment | Group |
| A | 0 | 28 | Vehicle Control | 4 |
| | | | EW-A-401 | 4 |
| B | 0, 4 | 28 | Vehicle Control | 4 |
| | | | EW-A-401 | 4 |
| C | 0 | 14 | Vehicle Control | 4 |
| | | | EW-A-401 | 4 |
| D | 0, 4 | 14 | Vehicle Control | 4 |
| | | | EW-A-401 | 4 |
| E | 0, 4, 8, 12 | 28 | Vehicle Control | 5 |
| | | | EW-A-401 | 5 |

Briefly, male Sprague Dawley rats (250 gm) were quarantined and acclimatized according to facility procedures. Rats were anesthetized using isoflurane. The area over the RF muscle was cleaned, shaved and prepared for surgery. Using aseptic techniques, a small incision was made in the skin, the muscle exposed, and the injection site was marked with a knotted suture, and vehicle or EW-A-401 was administered as a single injection. The needle was twisted as it was withdrawn to avoid extrusion of the test article. The skin was closed using glue or staples, and the animals allowed to recover. Animals that were dosed on subsequent days were similarly anesthetized, and injected at the same injection site (after palpation of the suture through the skin to identify the injection site and cleaning of the skin). Depending upon the test group, tissue was harvested either 14 or 28 days after the first dosing.

1 cm³ tissue sections, centered on the knotted suture were retrieved at sacrifice. These tissue cubes were trimmed, embedded in OCT, frozen in liquid Nitrogen and held at −80° C. until sectioning.

All animal work was approved by the Animal Care and Use Committee of the Biological Resource Facility at Edwards Lifesciences. Standard SOP's and procedures were used for animal husbandry, surgical procedures and euthanasia. Animals received humane treatment in an AALAC accredited facility.

### Histology and Immunohistochemistry

A series of frozen sections (6-10 um) were made from each tissue block. Slides were used immediately, or stored at −80° C. until used. Sections were stained for alkaline phosphatase activity (capillary density), immunostained for CD31 antigen (endothelial cell marker), or stained using hematoxalin and eosin (H&E).

### Capillary Density

Capillaries were quantified by counting alkaline phosphatase positive cells (AP+Cells) and number of muscle fibers in five random fields viewed at 100× magnification. Additionally, vascular density was calculated as the capillary to muscle fiber ratio.
6.3. Results
   6.3.1. Repeated administration of EW-A-401 leads to a more durable angiogenic response.

We have shown previously that single doses of EW-A-401 or vehicle are well tolerated in the normal hind limb. The above described results show that repeated administration of EW-A-401 leads to a more durable angiogenic response.

In this current study, daily cage-side observations were free of adverse findings. Inspections of the muscles at harvest were similarly free of gross findings.

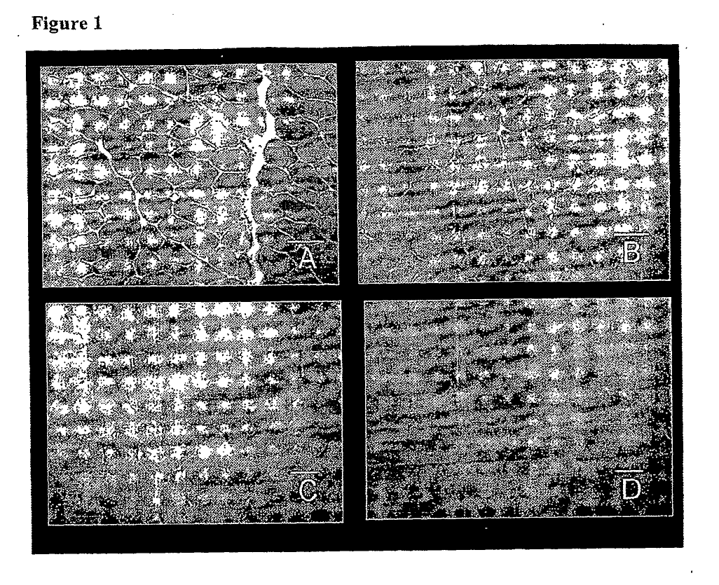

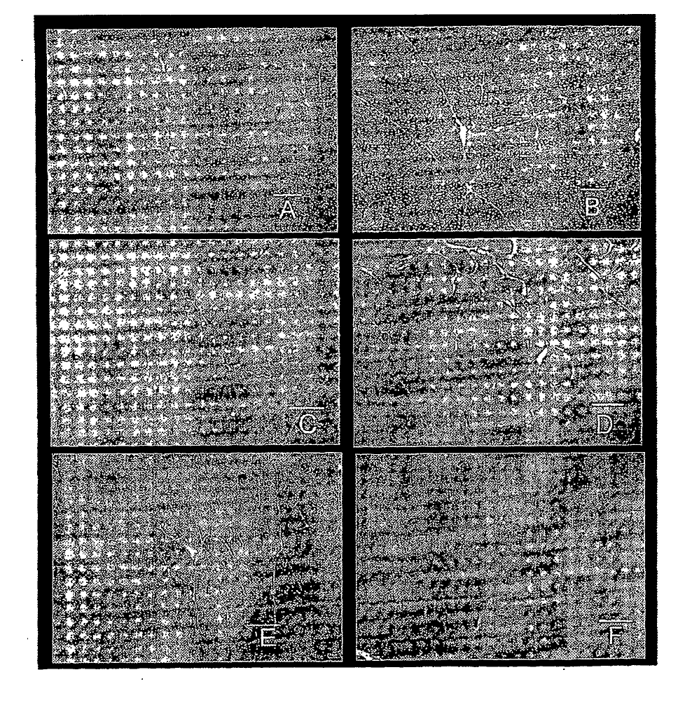

H&E sections of single and multiple doses of EW-A-401 or vehicle control (FIGS. 1A-D, FIGS. 2A-F) showed little interstitial swelling and a mild lymphocytic infiltrate observed in the injection site.

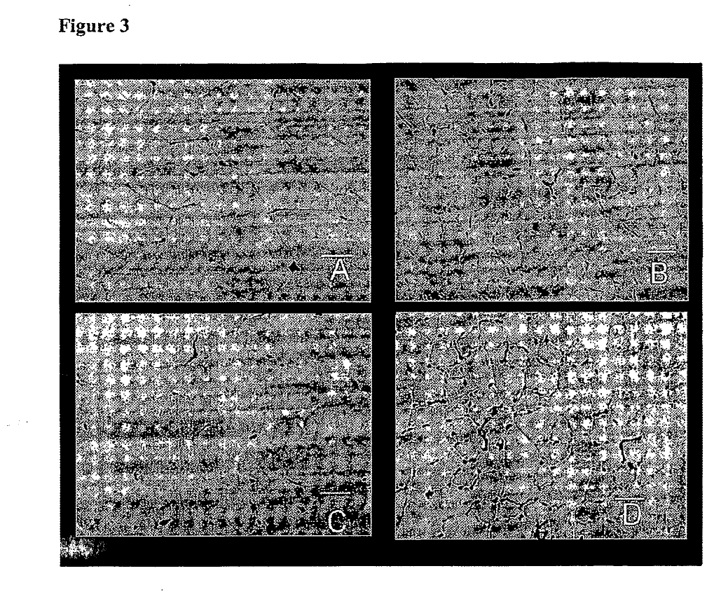

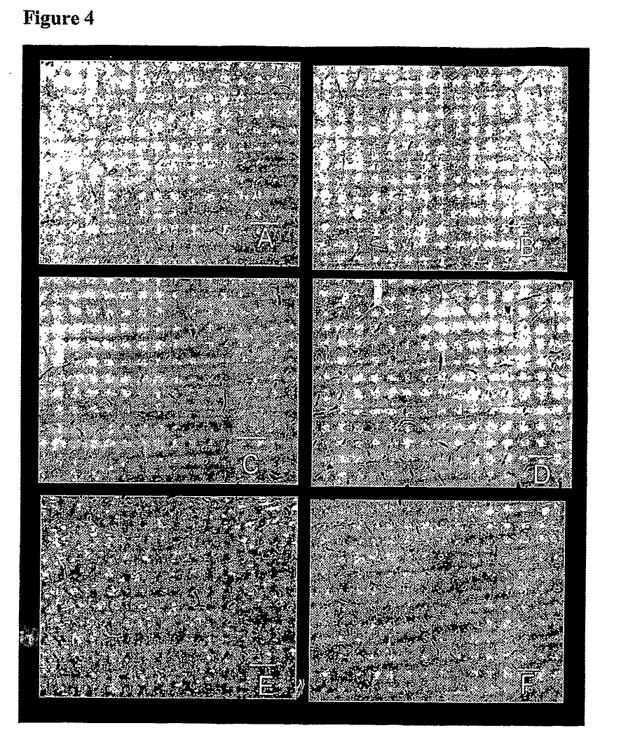

Alkaline phosphatase stained skeletal muscle sections from the vehicle control treatment groups were generally similar in appearance (FIGS. 3 A, C and FIGS. 4 A, C, E). This was irrespective of harvest day (14 or 28) or number of doses (1, 2, or 4).

Alkaline phosphatase stained skeletal muscle sections from the EW-A-401 treatment groups showed a general increase in capillary density compared to vehicle (FIGS. 3 B, D and FIGS. 4 B, D, F). Alkaline phosphatase staining was increased at 14 days in animals treated with one or two doses of EW-A-401, and remained increased at 28 days only in animals receiving multiple doses. The single dose group of EW-A-401 was similar to vehicle control at 28 days.

Selected sections were also stained with antibodies to CD 31, a second, independent marker for endothelial cells, using procedures described above. FIG. 5 shows that the pattern of staining of skeletal myocytes with CD 31 (FIG. 5 A, B) is similar to the pattern of staining with alkaline phosphatase (FIG. 5 C, D). This supports the conclusion that, as estimated by endothelial markers, capillary density is increased as a result of treatment with EW-A-401.

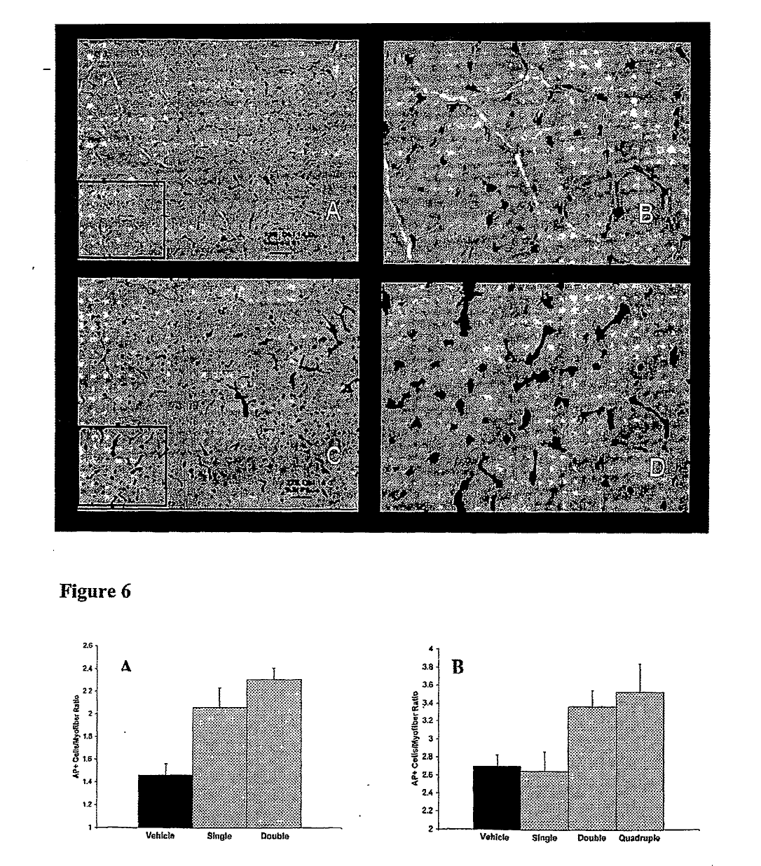

Capillary density was further evaluated by calculating the number of capillaries per myofiber in muscles harvested at 14 and 28 days; this data is presented in FIG. 6. At 14 days there was no statistical difference between the values for animals injected with one or two doses of vehicle (1.45±0.14 and 1.46±0.16 mean±SE, respectively) so these data were pooled for an average of 1.45±0.10 and are plotted in solid black (FIG. 6A). Similarly at 28 days there was no statistical difference between the values for animals injected with one, two, or four doses of vehicle control (2.42±0.165, 2.97±0.328, 2.70±0.08 mean±SE, respectively) so these data were pooled for an average of 2.69±0.12 and are plotted in solid black (FIG. 6B).

All animals groups injected with EW-A-401 at 14 days were significantly higher in capillary density than vehicle control (single dose=2.07±0.17, p<0.01, double dose=2.30±0.10, p<0.007) (FIG. 6A). At 28 days, double (3.36±0.178) or quadruple dose (3.53±0.31) groups of EW-A-401 were higher than the vehicle (2.69±0.12) and single dose (2.65±0.21) groups. Compared to vehicle control, the double dose group and the quadruple dose group were significantly higher in capillary density (p=0.006 and p=0.002, respectively). Data in FIG. 6 are presented as Mean±S.E, p values were determined with Student's t-test.
6.4. Conclusions

The results can be summarized as:
Capillary density
   In the rat hind limb at 14 days, capillary density increased in muscle from animals treated with single or two repeated doses of EW-A-401 relative to vehicle-treated controls.
   In the rat hind limb at 28 days, capillary density increased in muscles from animals receiving 2 or 4 repeated doses of EW-A-401 relative to vehicle-treated controls.

The implications of these observations for therapeutic applications include the conclusion that repeated administration of a ZFP enhances the therapeutic effect observed in peripheral disease.

The precise mode of action has not yet been determined, namely whether these responses to treatment with ZFP occur directly via transactivation by ZFP, or indirectly in response to VEGF-A or other genes upregulated in skeletal muscle by ZFP. In addition repeated administration of ZFP may cause an increase in the local concentration of the regulated genes, and/or increase the time course of expression of these regulated genes, leading to these phenotypic changes.

### 7. In Vivo Experiments According to the Invention—Example 2

7.1. Experimental Design and Objectives

The usefulness of the invention was shown by in vivo experiments to establish that
1. An Engineered VEGF-Inducing Transcription Factor Upregulates and Mobilizes Bone Marrow-Derived Progenitor Cells to Induce Angiogenesis in Peripheral Skeletal Muscle.

Therapeutic angiogenesis can provide a viable treatment strategy for a variety of vascular diseases. An engineered zinc finger transcription factor that induces expression of all isoforms of vascular endothelial growth factor (ZFP-VEGF) promotes angiogenesis in a rabbit model of hindlimb ischemia. However, it is not known whether this effect is mediated by vascular progenitor cells (VPCs). The following experiments were designed to evaluate the effects of ZFP-VEGF on the bone marrow (BM) reservoir of VPCs and their mobilization to peripheral blood in non-iscbemic mice.
7.2. Materials and Methods

Wild type C57BL/6 mice receive intramuscular (i.m.) delivery of either an empty plasmid as a control (days 0 and 3), a single injection of ZFP-VEGF plasmid (day 0), or two ZFP-VEGF plasmid injections (days 0 and 3).

Mice are sacrificed on day 7 (n=5) or 14 (n=5); the tibialis anterior muscle is harvested for analysis of capillary density and VEGF mRNA expression; and peripheral blood and bone marrow are harvested for analysis of lineage(lin)—c−kit+sca−1+VEGFR2+cells. To further characterize the effects of the ZFP-VEGF on BM-derived cells, transgenic mice expressing green fluorescent protein (GFP) under the CX3CR1 promoter (CX3CR1-GFP) are treated in a similar manner and sacrificed on day 7 (n=5) for analysis of effects on mononuclear and dendritic cells.
7.3. Results
   7.3.1. Repeated Administration of ZFP-VEGF Leads to Increased Angiogenic Response and Peripheral Blood VPCs.

Compared to control-treated mice, ZFP-VEGF treatment increased VEGF mRNA levels. A single injection of ZFP-VEGF had no effect on capillary density at 14 days, but two injections significantly increased capillary density (p<0.05 vs. control).

In contrast to the effects on vascularity, a single injection of ZFP-VEGF resulted in a >1 000-fold increase in BM VPC content, while two injections induced only a >10-fold increase in BM VPCs (both p<0.05 vs. control). However, two injections of ZFP-VEGF induced significant increases in peripheral blood VPCs compared to the control or single injection groups, while a single injection had no effect on blood VPC number. Consistent with these results, treatment of CX3CR1-GFP mice with two ZFP-VEGF injections resulted in significant increases in GFP+ cells in both peripheral blood and BM (p<0.05 vs control), however no GFP+ cells were detected in skeletal muscle at 7 days (3 days post-injection).
7.4. Conclusions

The results can be summarized as follows:
Treatment of non-ischemic skeletal muscle with
   a single i.m. injection of ZFP-VEGF plasmid induces expression of
      multiple VEGF isoforms
      increased BM VPC content
      does not mobilize VPCs to peripheral blood.
   Two i.m. injections of ZFP-VEGF plasmid upregulates
      BM VPCs
      dendritic or monocytic precursors
      does mobilize these cells to the peripheral blood circulation where they may contribute to sustained angiogenesis in non-ischemic muscle.

The implications of these observations for therapeutic application include the conclusion that ZFP-VEGF may provide a novel treatment for patients with atherosclerotic vascular disease.

The precise mode of action has not yet been determined.

These results suggest repeated localized administration of a zinc finger protein that stimulates VEGF to the same tissue can be useful in mobilizing vascular progenitor cells to the peripheral blood whereby they can be distributed to sites disseminated from the site of administration and treat disease at these sites. A site disseminated from the site of administration means a site whose cells do not receive the administered the zinc finger protein or nucleic acid directly from the administration but may be become populated by cells that do receive the administered zinc finger protein or nucleic acid as a result of migration of these cells from the site of administration to the disseminated site. Such administration is useful for treating diseases requiring stimulation of angiogenesis on a systemic basis or at least multiple disseminated sites, such as is the case with peripheral arterial disease, which can occur in any or all of the legs, arms or pelvis. In some methods, repeated administrations are made to the same site or sufficiently proximal sites that the zinc finger protein or nucleic acid being administered is delivered to a least some common cells among different administrations. The repeated delivery to the same cells promotes mobilization of vascular progenitor cells to the circulation.

All of the patents, publications and references cited herein are expressly hereby incorporated by reference in their entirety. Unless otherwise apparent from the context, any step, feature or element of the invention can be used in combination with any other.

1-4. (canceled)

5. The method of claim 9, wherein the repeatedly administering step comprises repeatedly administering the zinc finger protein or nucleic acid encoding the same by injection into a muscle of the patient.

6. The method of claim 5, wherein the zinc finger protein or nucleic acid encoding the same is repeatedly injected into the same muscle of the patient.

7. The method of claim 9, wherein the zinc finger protein or nucleic acid encoding the zinc finger protein is repeatedly injected at the same site or sufficiently proximal sites so that some cells receive repeated administrations of the zinc finger protein or nucleic acid.

8. The method of claim 5, wherein the muscle is non-ischemic.

9. A method of stimulating angiogenesis in a patient, comprising administering to the patient a zinc finger protein that induces expression of VEGF-A or a nucleic acid encoding the same-to stimulate angiogenesis at sites in the patient disseminated from a localized site of repeated administration.

10. The method of claim 9, wherein the zinc finger protein or nucleic acid encoding the same is formulated for intramuscular administration.

11. The method of claim 9 to treat peripheral arterial disease.

12. (canceled)

13. A method for treating diseases characterized by an impaired perfusion capacity of skeletal or cardiac muscle which comprises administering to a patient afflicted with said disease an effective amount of a zinc finger protein or a nucleic acid that encodes a zinc finger protein wherein said administering to a patient comprises the repeated administration of a therapeutic dosage to said patient.

14. The method according to claim 3, wherein said zinc finger protein or nucleic acid encoding the same is administered to said patient in a dosing regimen of repeated doses separated by intervals of at least one day.

15. The method according to claim 14, wherein the doses are separated by intervals of 3, 4, 7, or 10 days.

16. The method according to claim 14, wherein the doses are separated by an interval of 30 or more days.

17. The method according to claim 13, wherein each individual dose comprises 1 to 80 mg of said zinc finger protein or nucleic acid encoding said zinc finger protein.

18. The method according to claim 13, wherein said individual dose comprises 2 mg/ml of said zinc finger protein or nucleic acid.

19. The method according to claim 13, wherein said patient is administered a total of 2 to 10 dosages.

20. The method according to claim 13, wherein said zinc finger protein binds to a target site of a nucleic acid that modulates expression of a VEGF gene.

21. The method according to claim 13, wherein the zinc finger protein binds to a target site comprising the sequence GGGGGTGAC.

22. The method according to claim 13, wherein said zinc finger protein comprises at least three fingers having the sequence DRSNLTR, TSGHLTR and RSDHLSR.

23. The method according to claim 13, wherein said zinc finger protein is fused to a regulatory domain.

24. The method according to claim 13, wherein said nucleic acid is contained within an expression vector or cassette operably linked to a promoter.

25. The method according to claim 24, wherein said expression vector or cassette further comprises a nuclear localization sequence and an activation domain.

26. The method according to claim 24, wherein said expression vector is a viral expression vector.

27. The method according to claim 26, wherein said viral expression vector is a pVax vector, an adenoviral expression vector or an AAV expression vector.

28. The method 13, wherein said nucleic acid is administered in the form of a plasmid.

29. The method according to claim 28, wherein said nucleic acid is administered in the form of naked DNA or RNA.

30. The method according to claim 28, wherein said nucleic acid is administered in association with viral proteins, viral polypeptides, viral capsid proteins or a cell transfer/uptake enhancing agent.

31. The method according to claim 30, wherein said cell transfer/uptake enhancing agent is poloxymer.
